(19) United States
(12) Patent Application Publication
(10) Pub. No.: US 2009/0246816 A1
(43) Pub. Date: Oct. 1, 2009

(54) METHODS AND KITS FOR SELECTING AN ANALGESIC REGIME

(76) Inventor: Malcolm Knox, Lancashire (GB)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/374,400

(22) PCT Filed: Jul. 20, 2007
(86) PCT No.: PCT/GB07/02766

§ 371 Date: Apr. 24, 2009

(30) Foreign Application Priority Data
Jul. 20, 2006 (GB) 0614396.0

(51) Int. Cl. C12Q 1/26

(52) U.S. Cl. 435/25

(57) ABSTRACT

A method is provided for selecting an analgesic regime for treatment of a patient. In the method, a body fluid sample from the patient is assayed for the presence of a metabolite produced by metabolism of a prodrug. If the metabolite is present in the body fluid sample this indicates that a suitable analgesic regime for the patient should comprise administration of at least one analgesic agent selected from the group consisting of: codeine and tramadol. If the metabolite is absent from the body fluid sample indicates that a suitable analgesic regime for the patient should comprise administration of an analgesic agent selected from the group consisting of: dihydrocodeine; buprenorphine and propoxyphene. The invention also provides a kit for selecting an analgesic regime for a patient. The kit comprises means for assaying for the presence of a metabolite produced by metabolism of a prodrug and instructions for using the provided means to perform the method as described above.

The present invention relates to methods for selecting an analgesic regime for a patient. The invention also provides kits for selecting an analgesic regime for a patient. The invention further provides a method for detecting the presence of an enzyme of interest in a human or an animal body, as well as a kit for the detection of an enzyme of interest.

Pain is a major cause of discomfort, agitation and anxiety for patients. Pain may also lead to several important complications in addition to being unpleasant. These complications may be severely damaging, and in extreme cases may even lead to the death of a patient. For example, it has been suggested that certain instances of the morbidity and mortality related to surgery and trauma may be the result of the pathological disturbances initiated by severe acute pain, or acute pain that is not treated with an appropriate analgesic regime.

Pain may be defined as “an unpleasant sensory and emotional experience associated with actual or potential tissue damage or described in terms of such damage”, a definition initially put forward by the International Association for the Study of Pain in 1979. Analgesia is the alleviation of pain, and for the purposes of the present specification should be considered to be achieved by pharmaceutical means. Thus analgesic drugs are drug compounds provided for the reduction of pain, and analgesic regimes are treatment regimes designed to provide pain relief for a patient.

Pain is primarily a protective mechanism, and its purpose may be thought of as bringing attention to the fact that tissue damage is occurring or about to occur. Pain is accompanied by motivated behavioural responses, such as withdrawal or defence, as well as emotional reactions, such as crying or fear. Past experiences, the patient's attitudes, beliefs, and personality can also shape the subjective perception of pain.

Pain, and the patient's subjective perception of pain, may contribute to a number of deleterious effects that may limit the treatment that can be provided for existing damage, and may even cause further damage themselves. By way of example, patients with chest wall or abdominal incisions who do not receive adequate acute pain control have limited ability to take a deep breath, cough, and tolerate chest physiotherapy. This causes sputum retention and atelectasis, increasing the risk of pneumonia, hypoxaemia and death. Pain also limits mobilisation, which may restrict participation in physiotherapy and rehabilitation, leading to longer hospital stay. Such limited mobilisation can also lead to complications of immobility, such as deep vein thrombosis and pressure sores.

Furthermore, pain causes a number changes in the body of a patient experiencing pain. The haemodynamic responses to acute pain are tachycardia and hypertension, which increase myocardial oxygen consumption. These lead to coronary ischaemia, and this is implicated in the development of myocardial infarction associated with incidences of pain.

Management of patient pain using an appropriate analgesic regime provides important benefits and may alleviate some, or all, of the disadvantages described above. One widely-adopted approach to the treatment of pain is the WHO (World Health Organisation) “analgesic ladder”, which was first put forward in 1986 for the relief of pain associated with cancer, and has since been utilised by clinical practitioners the world over. The key principles underlying the WHO analgesic ladder are that pain relief should generally be “by mouth” (reflecting a preference for use of orally administered analgesics), “by the clock” (referring to the administration of analgesics at set intervals, typically three to six hours, rather than on demand), “by the ladder” (a reference to the prescribing guidelines associated with various steps of the ladder), and “for the individual” (reflecting the fact that different individuals react to pain in different manners, and that different patients have different responses to different analgesic drugs).

The WHO analgesic ladder utilises three “steps” or “rungs” that broadly correspond to three increasing levels of pain experienced by a patient (from mild pain in step one, through moderate pain in step two, to severe pain in step three), or to changes in function or quality of life experienced by a patient. The WHO analgesic ladder provides guidance as to analgesic drugs, and optionally adjuvants, that may be prescribed to a patient experiencing pain of the specified intensity. In the event that a patient does not experience pain relief on one step of the analgesic ladder, they should progress to the next step.

“Analgesic drugs”, considered in the WHO analgesic ladder, are those drugs administered solely for the reduction of pain. Examples of analgesic drugs that may be used in accordance with the ladder include non-opiate analgesics (such as paracetamol) or opiate analgesics.

“Adjuvants” in the context of the WHO analgesic ladder are compounds that are not primarily analgesic in their methods of action (although they may have analgesic effects in certain contexts), but that are administered for other purposes. Suitable adjuvants to be used in accordance with the WHO analgesic ladder include agents for the reduction of inflammation, such as nonsteroidal anti-inflammatory drugs (NSAIDs), antiemetic agents, laxatives, anti-diarrhoeal agents, antidepressants, antipsychotics, anticonvulsants, cortiosteroids, anxiolytics, and psychostimulants. Suitable adjuvants may be selected on the basis of their ability to reduce opiate side effects such as nausea.

Patients on the first step of the WHO analgesic ladder are to be prescribed an analgesic regime involving provision of non-opioid analgesics, such as paracetamol. These non-opioid analgesic drugs may be provided in combination with an adjuvant such as aspirin or another suitable NSAID.

Patients on the second step of the ladder are to be treated with an analgesic regime involving provision of a “weak opioid” analgesic. This weak opioid may be provided in combination with a suitable adjuvant, for example an NSAID as described before or an antidepressant or anticonvulsant adjuvant in the case of a patient suffering from neuropathic pain.

Finally, a patient on the third step of the WHO analgesic ladder is to be prescribed an analgesic regime involving provision of a “strong opioid” analgesic drug. The strong opioid may be provided with an adjuvant in order to reduce side effects caused by the opioid, or to provide other beneficial effects.

“Weak opioids” suitable for use in step two of the WHO analgesic ladder include codeine, dihydrocodeine, propoxyphene, tramadol, thebaine, pentazocine, buprenorphine and meptazinol. Weak opioids are also sometimes referred to as “opioids for mild to moderate pain”. Weak opioids may be considered to be those that act as partial agonists at opioid receptors.

“Strong opioids” suitable for use in step three of the WHO analgesic ladder include alfentanil, diamorphine, hydromorphone, levorphanol, morphine, methadone, oxycodone, pethidine, phenazocine, remifentanil, and sufentanil. Strong opioids are sometimes referred to as “opioids for mild to moderate pain”. Strong opioids may be considered to be those that act as full agonists at opioid receptors.

It will be appreciated that references to therapeutic compounds in the context of the present disclosure should, save for where the context requires otherwise, be taken to comprise any suitable preparation comprising the therapeutic compound. Merely by way of example references to morphine should be taken also to comprise: oromorph, sevredol, morcap, Morphine Sulphate Tablets (MST), MST continues, Zomporph, MXL, Moraxen. Furthermore, for purposes of brevity, British nomenclature has been used, consistent with that used in the British National Formulary. The identity of compounds and formulations referred to will thus be readily understood by the skilled practitioner based on these disclosures.

The WHO analgesic ladder has been well received, and extensively adopted. Validation studies suggest that the ladder may be used to provide pain relief to between 77% and 100% of advanced cancer patients otherwise experiencing pain.

Despite the wide-spread adoption of the WHO analgesic ladder, there are areas where it is recognised that the existing ladder may be improved and thereby benefit patients. It is known that the responses of individual patients to opioid analgesics vary widely. This is particularly the case in response to “weak opioids” such as those used in step two of the analgesic ladder. The variations that exist are so severe that some patients may not obtain pain relief when provided with certain analgesic drugs that are otherwise effective for relief of pain in other patient groups. Alternatively variations may mean that certain patients are more prone to side effects that render use of particular analgesic drugs problematic.

Currently a range of opioid drugs, and particularly weak opioid drugs, are used in an effort to overcome these problems. However, there remains a well recognised need for methods by which suitable analgesic regimes for the treatment of patients may be selected. Indeed, in an interview conducted as part of an appraisal of the WHO analgesic ladder on its twentieth anniversary, Dr. Kathleen Foley, former chair of the WHO Expert Committee on Cancer Pain Relief and Active Supportive Care, noted “there is an enormous need for . . . studies to address which patients respond to what drugs and at what doses”.

Accordingly, it is an aim of certain embodiments of the present invention to provide methods by which an analgesic regime may be selected for use in patients that do not experience pain relief through administration of all weak opiate analgesic drugs. It is an aim of certain embodiments of the invention to provide kits by which an analgesic regime may be selected for use in patients that do not experience pain relief through administration of all weak opiate analgesic drugs.

In a first aspect, the present invention provides a method for selecting an analgesic regime for treatment of a patient, the method comprising assaying a body fluid sample from the patient for the presence of a metabolite produced by metabolism of a prodrug;
i) wherein presence of the metabolite in the body fluid sample indicates that a suitable analgesic regime for the patient should comprise administration of at least one analgesic agent selected from the group consisting of: codeine and tramadol; and
ii) wherein absence of the metabolite in the body fluid sample indicates that a suitable analgesic regime for the patient should comprise administration of an analgesic agent selected from the group consisting of: dihydrocodeine; buprenorphine and propoxyphene.

It may generally be preferred that an analgesic regime in accordance with ii) above comprises administration of dihydrocodeine.

In a second aspect, the present invention provides a kit for selecting an analgesic regime for a patient, the kit comprising:
i) means for assaying for the presence of a metabolite produced by metabolism of a prodrug; and
ii) instructions for using the provided means to perform the method as described in any embodiment of the first or third aspects of the invention.

The methods and kits of the invention allow the selection of an appropriate analgesic regime for the relief of pain in a patient. An appropriate regime may be one that is best suited to reduce pain that would otherwise be experienced by the patient. As noted above, there is a well established need for methods and kits that may be used to provide improved pain relief for patients by allowing the selection of appropriate analgesic regimes to be used in the management of individual patients' pain.

The methods and kits of the invention allow identification of patients having impaired metabolism of codeine and/or tramadol, and who thus do not obtain the full benefit of pain relief caused by products of codeine and/or tramadol metabolism. Patients who have such impaired metabolism of codeine and/or tramadol may obtain beneficial pain relief through the use of analgesic regimes utilising other opiate analgesics such as dihydrocodeine. The methods and kits of the invention allow the identification of such patients and the rapid selection of appropriate analgesic regimes able to effectively reduce the level of pain experienced.

Use of the methods and kits of the present invention provides notable advantages to patients requiring pain relief, compared to existing approaches by which analgesic regimes may be adopted. Not only does the use of the methods or kits of the invention help to avoid patients being treated with unsuccessful regimes during which pain will continue to be experienced (even if only briefly), but they also help to avoid unnecessary use of strong opioid analgesics (such as on elevation to step three of the WHO analgesic ladder) by allowing selection of appropriate weak opioid-based regimes (based on use of codeine and/or tramadol, or use of dihydrocodeine) able to alleviate a patient's pain.

These advantages are considered in greater detail below.

In existing methods known from the prior art, such as the well established WHO analgesic ladder, selection of a suitable analgesic strategy is achieved by a process of “trial and error” in which failure of a selected regime to alleviate pain leads to the selection of an alternative analgesic regime. It will immediately be recognised that patients treated with ineffective analgesic regimes during such a process will remain subject to pain, and hence to the effects of pain. Existing methods for the selection of analgesic regimes may involve prolonged periods in which a patient is not receiving effective analgesia, and this is associated with many deleterious effects on the patient.

Instead of the previous approach based on trial and error, the methods and kits of the invention allow an informed decision to be taken as to the selection of an appropriate analgesic strategy, this decision being based on individual properties of the patient that determine the patient's response to different analgesic drugs. Although the WHO analgesic ladder has been in existence, and widely adopted, since 1986, it has not previously been suggested that the ladder be modified in the manner contemplated in the present invention, or to attain the results (a “tailoring” of analgesic strategies for an individual patient) described herein.

The methods and kits of the invention also provide a notable advantage in that they allow effective analgesic regimes able to alleviate pain in a patient to be selected based on the use of appropriate weak opioids, particularly dihydrocodeine. This approach is thus able to avoid unnecessary escalation to strong opioids in the case of patients able to attain pain relief through the use of an appropriate weak opioid.

However, it will be appreciated that the progression of analgesic regimes (such as those set out in points i) and ii)) to incorporate optional use of strong opioids (such as those selected from the group consisting of: alfentanil, diamorphine, hydromorphone, levorphanol, morphine, methadone, oxycodone, pethidine, phenazocine, remifentanil, and sufentanil) is not precluded from the methods of the invention.

It will be appreciated that the methods or kits of the invention may be used for the selection of an analgesic regime for the treatment of a patient already experiencing pain, or may be used prophylactically, i.e. for the selection of an appropriate analgesic regime for a patient prior to that patient experiencing pain. In this latter embodiment, in which the methods or kits of the invention are used prophylactically, the ability to identify whether a patient will receive the greatest analgesic effect from a regime based on the use of codeine and/or tramadol, or whether greater analgesic effect will be derived from a regime based on the use of dihydrocodeine, may allow the appropriate regime to be implemented immediately at a later point when the patient has a need for analgesia utilising weak opioids (i.e. without the need for the method or kit to be used at a time when the patient is experiencing pain). Information derived from the prophylactic use of such methods or kits may be entered on a patient's records for future use.

It will be appreciated that methods and kits of the invention may be practiced with reference to a number of different prodrugs, and hence a number of different metabolites. Typically the prodrug may be a weak opioid, as considered elsewhere in the specification. In a preferred embodiment the prodrug may be codeine. In the case that the prodrug is codeine, the metabolite to be assayed for may be one or more compounds independently selected from the group consisting of morphine, morphine 3 gluceronide, and morphine 6 gluceronide.

In a further preferred embodiment the prodrug may be tramadol. In the case that the prodrug is tramadol, the metabolite to be assayed for may be O-desmethyltramadol, a compound also referred to as M1.

The method of the invention may further comprise the step of administering the prodrug to the patient prior to conducting the assay. This ensures that the patient receives sufficient of the prodrug present to allow a detectable amount of the metabolite to potentially accumulate in the body fluid sample to be assayed. In the event that the method of the invention comprises the step of administering the prodrug to the patient, the time elapsing between administration of the prodrug and assaying for the metabolite will preferably be four days or less, and more preferably two days or less, even more preferably one day or less, yet more preferably twelve hours or less, and may be six hours or less. In a preferred embodiment the elapsed time may be between six and twelve hours.

Generally it may be preferred that the time elapsing between administration of the prodrug and assaying for the metabolite will be 30 minutes or greater. In the case that codeine is the prodrug administered it may be preferred that the time elapsed is an hour or more, and in the case that tramadol is the prodrug administered it may be preferred that the time elapsed is two hours or more.

In embodiments of the method of the invention that include administration of the prodrug prior to assaying for its metabolites, the prodrug may preferably be administered in an amount that is not sufficient to provide an analgesic effect (i.e. an amount below the recommended dosage that would be provided for analgesia). The inventor believes that the use in this manner of a dose of the prodrug that is below the analgesic level also provides the notable advantage that a suitable analgesic regime may be selected without risking induction of side effects in the patient that is to be treated.

A suitable amount of the prodrug to be administered in methods in accordance with this embodiment of the invention may be determined with reference to the specific prodrug selected. However, by way of guidance, in the event that codeine is the selected prodrug, a suitable amount to be administered in accordance with this embodiment may be between approximately 1 mg and 4 mg. By way of further guidance, in the event that tramadol is the selected prodrug, a suitable amount to be administered in accordance with this embodiment may be below about 30 mg, and may preferably be between approximately 5 mg and 15 mg.

The skilled person will appreciate that the analgesic regime referred to in i) (the analgesic regime to be adopted in the case that the metabolite is present in the body fluid sample) or the analgesic regime referred to in ii) (the analgesic regime to be adopted in the case that the metabolite is absent from the body fluid sample) may further comprise administration of a non-opioid analgesic, such as paracetamol. An analgesic regime selected in accordance with the present invention may preferably comprise the oral administration of analgesic compounds (be they codeine and/or tramadol; or dihydrocodeine). An analgesic regime selected in accordance with the present invention may further comprise provision of an adjuvant, such as agents for the reduction of inflammation (e.g. nonsteroidal anti-inflammatory drugs), antiemetic agents, laxatives, anti-diarrhoeal agents, antidepressants, antipsychotics, anticonvulsants, cortiosteroids, anxiolytics, and psychostimulants.

It may be preferred that, optionally or additionally, the analgesic regime referred to in ii) (the analgesic regime to be adopted in the case that the metabolite is absent from the body fluid sample) further comprises the administration of tramadol to the patient.

The methods or kits of the invention may preferably be used for the selection of an analgesic regime for alleviation of pain associated with cancer. However, it will be appreciated that the methods or kits of the invention may be utilised for the selection of an appropriate analgesic regime in any context where weak opiates are to be used in the alleviation of a patient's pain. The inventor believes that the methods or kits of the invention may be used in the selection of appropriate analgesic regimes for the treatment of all nociceptive, neuropathic and mixed pain aetiologies. For example, the inventors believe that the methods or kits of the invention may be used to select appropriate analgesic regimes for the alleviation of post-operative pain, pain associated with chronic wounds, pain associated with osteoarthritis, back pain, headache, or neck pain.

The body fluid sample assayed for the presence of the metabolite may be selected from the group consisting of: a urine sample; a saliva sample; a blood sample; a plasma sample; a lymph sample; and a cerebrospinal fluid sample. It may generally be preferred that the body fluid sample to be assayed is a urine sample.

The methods of the invention may optionally comprise a further step of obtaining a body fluid sample from a patient. The kits of the invention may further comprise means for collecting a body fluid sample from a patient, or means for containing such a sample prior to and/or during an assay. In the case where it is wished to use a blood sample as the body fluid sample such means may comprise lancets, needles suitable for phlebotomy and/or evacuated, or partially-evacuated, containers (of the sort sold under the Registered Trade Mark “Vacutainer®”). In the case where it is wished to utilise a urine body fluid sample, suitable means may comprise containers for the collection of urine.

Many suitable techniques by which the assay for the metabolite is may be conducted are known to those skilled in the art. By way of example, a suitable assay may be performed by means of a reactive test device of the sort described in U.S. Pat. No. 3,901,657. These devices are able to convert morphine to a chromogenic compound, and thereby indicate the presence of this metabolite in a body fluid sample. Briefly, the devises comprise a first portion which includes a morphinophilic agent (such as a cationic exchange resin) and a second portion which includes a periodate reagent for morphine, this agent being capable of reacting with morphine to produce a chromogenic compound that gives rise to a visually observable colour product.

U.S. Pat. No. 3,901,657 describes the manufacture and use of such reactive test devices in some detail, and the contents of this disclosure are incorporated herein by reference, particularly to the extent that they describe manufacture or use of such devices. Merely by way of example, the disclosure of U.S. Pat. No. 3,901,657 suggests that a suitable periodate reagent for use in such reactive test devices may comprise a pyridine in combination with a compound selected from the group consisting of paraperiodic acid and its soluble salts and esters.

It will be appreciated that reactive test devices, and particularly those of the type described above and in U.S. Pat. No. 3,901,657, constitute preferred means by which a body fluid sample may be assayed for the presence of a metabolite in accordance with the methods of the invention, and represent preferred means for assaying for the presence of a metabolite produced by metabolism of a prodrug for use in kits in accordance with the invention.

Immunoassays, such as enzyme linked immunosorbent assays (ELISAs) or radioimmunoassays, constitute a further preferred embodiment by which fluid samples may be assayed for the presence of metabolites. Suitable immunoassays may be undertaken in respect of any metabolite for which an immunological binding partner exists. Examples of suitable immunological binding partners include monoclonal antibodies, polyclonal antibodies, and fragments of such antibodies capable of binding to a chosen metabolite.

Merely by way of example, and without limitation, the presence of the metabolite morphine in a body fluid sample may be detected using an immunoassay using any one of a number commercially available antibodies, including those selected from the group consisting of: goat anti-morphine polyclonal antibody sold by Abcam; mouse anti-morphine monoclonal antibody clone 3A6 sold by Abcam; mouse anti-morphine monoclonal antibody clone BDI263 sold by Abcam; mouse anti-morphine monoclonal antibody clone BDI918 sold by Abcam; rabbit anti-morphine polyclonal antibody sold by Abcam; sheep anti-morphine polyclonal antibody sold by Abcam; mouse anti-morphine monoclonal antibody clone MOR-001 sold by AbD Serotec; mouse anti-morphine monoclonal antibody clone 6D6 sold by Aviva Systems Biology; mouse anti-morphine monoclonal antibody clone 094-10034 sold by Meridian Life Science, Inc.; mouse anti-morphine monoclonal antibody clone BDI297 sold by Meridian Life Science, Inc.; and mouse anti-morphine monoclonal antibody clone 201 sold by Santa Cruz Biotechnology, Inc.

In the event that it is wished to utilise an immunoassay to assay for the presence of a metabolite in a body fluid sample, kits of the invention may comprise an antibody, or antibody fragment, capable of binding to an appropriate metabolite. Kits of the invention may optionally further comprise plasticware for use in an immunoassay, and/or other reagents useful for the practice of imnmunoassays such as buffer solutions, means by which antibodies may be labelled (e.g. enzymes such as horseradish peroxidise), and means by which labelled antibodies may be detected (e.g. chromogenic substrates of suitable enzymes).

Instructions for use to be provided as part of a kit of the invention may be provided in the form of written instructions, or may be provided in other suitable forms, such as computer-readable instructions.

A kit according to the present invention may further comprise an amount of one or more prodrugs. A suitable prodrug may, for example, be selected from the group consisting of: codeine and tramadol. A prodrug to be provided as part of a kit of the invention may preferably be provided in the form of discrete dosage units. Preferably each dosage unit may comprise an amount of the prodrug that will not give rise to the development of side effects. This may typically be below the amount required to bring about an analgesic effect.

In the case of a kit comprising an amount of the prodrug codeine, the prodrug may be provided in dosage units comprising between approximately 1 mg and approximately 4 mg of the prodrug. A total amount of up to 120 mg of the prodrug may be provided in a single kit.

In the case of a kit comprising an amount of the prodrug tramadol, the prodrug may be provided in dosage units comprising less than 30 mg and preferably between approximately 5 mg and 15 mg of the prodrug. A total amount of up to 150 mg of the prodrug may be provided in a single kit.

In a further embodiment, the present invention provides the use of a means for detecting a metabolite produced by metabolism a prodrug in the manufacture of a kit for the selection of an analgesic regime for a patient.

In the case of either methods or kits in accordance with the present invention, the metabolite to be assayed for may preferably be formed by metabolism of the prodrug via the action of a cytochrome P450 enzyme. It may be preferred that the cytochrome P450 enzyme is cytochrome P450 2D6.

Indeed, so important is the role of cytochrome P450 2D6 that it gives rise to a third aspect of the invention, which provides a method for selecting an analgesic regime for treatment of a patient, the method comprising assaying a body fluid sample from the patient for the presence of a metabolite produced by metabolism of a prodrug by cytochrome P450 2D6;
i) wherein presence of the metabolite in the body fluid sample indicates that a suitable analgesic regime for the patient should comprise administration of a prodrug metabolised by cytochrome P450 2D6 to yield an analgesic drug; and
ii) wherein absence of the metabolite in the body fluid sample indicates that a suitable analgesic regime for the patient should comprise administration of a drug having intrinsic analgesic activity.

In the case of an assay in accordance with the third aspect of the invention any suitable prodrug metabolised by cytochrome P450 2D6 to yield an analgesic drug may be used in an appropriate analgesic regime (as referred to in i) above). Preferred examples of such prodrugs include codeine and/or tramadol. Both codeine and tramadol also represent examples of prodrugs that are metabolised by cytochrome P450 2D6 to yield an analgesic drug. Suitable drugs having intrinsic analgesic activity will be drugs that do not have to undergo metabolism by cytochrome P450 2D6 in order to yield an analgesic drug. Examples of such drugs having intrinsic analgesic activity include dihydrocodeine, propoxyphene, buprenorphine and “strong opioids” such as those considered elsewhere in the specification.

In a fourth aspect, the invention further provides a method for selecting an analgesic regime for treatment of a patient, the method comprising assaying a sample representative of gene expression in the patient for an expression product representative of expression of cytochrome P450 2D6;
i) wherein presence in the sample of the expression product representative of expression of cytochrome P450 2D6 indicates that a suitable analgesic regime for the patient should comprise administration of codeine and/or tramadol; and
ii) wherein absence in the sample of the expression product representative of expression of cytochrome P450 2D6 indicates that a suitable analgesic regime for the patient should comprise administration of an analgesic agent selected from the group consisting of: dihydrocodeine; buprenorphine and propoxyphene.

A suitable sample to be used in an assay in accordance with this aspect of the invention may be any sample that contains information representative of gene expression in the patient. A preferred sample of this type may be a sample containing cells from which information regarding gene expression may be derived. Suitable cells may be obtained by means of a swab, and particularly by means of an oral swab, in which cells from the cheek lining or the like may be collected.

An expression product representative of expression of cytochrome P450 2D6 may comprise a polypeptide, such as cytochrome P450 2D6 itself, or a nucleic acid molecule, for example mRNA, encoding cytochrome P450 2D6.

A suitable assay for the presence of an expression product representative of the expression of cytochrome P450 2D6 may be readily selected with reference to the nature of the expression product. For example, in the case that the expression product is a polypeptide, such as cytochrome P450 2D6, the assay may use an immunoreactive reagent capable of binding specifically to the polypeptide. By way of example, a suitable reagent may comprise an antibody, or antibody fragment, capable of binding to cytochrome P450 2D6 such as one selected from the group consisting of: rabbit anti-cytochrome P450 2D6 polyclonal antibody (sold by Abcam); mouse anti-human cytochrome P450 2D6 monoclonal antibody clone 512-1-8 (sold by MBL International); and goat anti-human cytochrome P450 2D6 polyclonal antibody (sold by Santa Cruz Biotechnology, Inc.).

It will be appreciated that such antibodies or antibody fragments may be used in any suitable immunoassay, including immuno-blotting (Western blotting); radio-immunoassays; ELISAs; or the like.

In the event that the expression product representative of expression of cytochrome P450 2D6 is a nucleic acid molecule, such as mRNA, the assay may use specific amplification of the nucleic acid molecule. Merely by way of example, such an assay may use primers specific for nucleic acids encoding cytochrome P450 2D6 in a polymerase chain reaction (PCR) to detect the presence of the nucleic acid expression product.

In a fifth aspect, the present invention provides a kit for selecting an analgesic regime for treatment of a patient, the kit comprising:
i) means assaying a sample representative of gene expression in the patient for an expression product representative of expression of cytochrome P450 2D6; and
ii) instructions for using the means provided to perform a the method as described in any embodiment of the previous aspect of the invention.

A kit in accordance with this aspect of the invention may, for instance comprise an antibody, or antibody fragment, capable of binding specifically to a polypeptide expression product (such as cytochrome P450 2D6 itself). Alternatively or additionally, a kit in accordance with this aspect of the invention may comprise primers for the amplification of a nucleic acid expression product.

In a sixth aspect, the invention provides a method for detecting the presence of an enzyme of interest in a human or an animal body, which method comprises:
i) administering to a human or animal subject a prodrug or other chemical entity capable of being converted by the enzyme to another drug or chemical entity, and
ii) detecting in the urine or other bodily fluid the direct or subsequent metabolic product of enzymatic interaction with the administered entity.

In a seventh aspect the invention provides, a kit for the detection of an enzyme of interest, the kit comprising:
i) a prodrug or other suitable chemical entity, and
ii) a device for detecting the presence of the derivable drug or the presence of a specific altered chemical entity in the urine or other bodily fluid whose existence in the human or animal body depends on the combination of the presence of the enzyme and the administered chemical entity

The methods or kits of the sixth or seventh aspects of the invention give may be used in tests for cytochrome P450 2D6, a preferred example of an “enzyme of interest” in accordance with these aspects.

Cytochrome P450 2D6 metabolises many compounds, including Amitriptyline, Captopril, Clomipramine, Clozapine, Codeine, Desipramine, Dextrromethorphan, Flecainide, Fluoxetine, Haloperidol, Hydrocodone, Imipramine, Metoprolol, Mexilitine, Nortriptyline, Ondansetron, Oxycodone, Paroxetine, Perphenazine, Propafenone, Propoxyphene, Propranolol, Quinidine, Risperidone, Ritanovir, Sertraline, Thioridazine, Timolol, Tramadol and Venlafaxine. Any one, or more, of these compounds may be used as a prodrug in a method or kit in accordance with the fifth or sixth aspects of the invention.

It is possible to test for the activity of this cytochrome P450 2D6 by administering any of the drugs prodrugs listed above, and then assaying for a specific metabolic product formed by the action of the enzyme on the administered prodrug.

A preferred embodiment of a method in accordance with the sixth aspect of the invention uses codeine as the prodrug and morphine as the product of metabolism, detection of the presence of which in turn indicates the presence of an active form of the enzyme cytochrome P450 2D6.

Such a method may be practiced by administration of codeine in a dose of at least 0.001 milligrams and up to 30 grams or more, followed by an assay for the presence of morphine in the urine or other bodily fluid. Alternatively the method may involve assaying for morphine 3 gluceronide or morphine 6 gluceronide in urine or other bodily fluid.

Most preferably, codeine is administered in a dose of between 1 mg and 360 mg, more preferably between 1 mg and 4 mg, in divided doses of one quarter the total to be administered, and this divided dose administered four times a day. Administration of this sort establishes a steady plasma level over a period of several hours to a day. At the end of the selected time morphine is assayed for in the urine or other bodily fluid. The alternate method would involve assaying for morphine 3 gluceronide or morphine 6 gluceronide in urine or other bodily fluid.

An alternative method may involve the administration of tramadol in a dose of at least 0.001 milligrams and up to 50 grams, more preferably in a dose between 5 mg and 15 mg. O-desmethyltramadol (M1) may then be assayed for in the urine or other bodily fluid. Alternatively the method may involve assaying for gluceronide derivatives of (M1) in the urine or other bodily fluid.

Preferably, tramadol is administered in a dose of 5 mg to 400 mg, more preferably 5 mg to 15 mg, in divided doses of one third of the total dose and administered 3 times a day over a period of several hours to a day and O-desmethyltramadol is assayed for in the urine or other bodily fluid. Alternatively the method may involve assaying for morphine 3 gluceronide or morphine 6 gluceronide in urine or other bodily fluid.

Methods or kits in accordance with the sixth or seventh aspects of the invention may be used in tests for cytochrome P450 1A2.

Such tests may use a similar method to those described above, but instead the prodrug should comprise a substrate of cytochrome P450 1A2. Suitable examples of such substrates include Amitriptyline, Caffeine, Clomipramine, Claozapine, Ethinylestradiol, Imipramine, Olanzapine, Paracetamol, Propranolol, Theophyline, Trimipraine (all names relate to chemical structural chemical entities as understood in the British National Formulary number 50 Sept 2005).

A test for the presence of cytochrome P450 1A2 using a method or kit in accordance with the sixth or seventh aspect of the invention may be based on the use of one or more of the prodrugs described above. All of these undergo 3 demethylation by cytochrome P450 1A2, and this gives rise to the metabolites to be assayed for.

In a preferred embodiment, caffeine is administered to a subject in a dose of 0.001 mg to 10 grams and a device used to detect the 3 demethylation product produced by caffeine's interaction with the enzyme. Most preferably a dose of 80-160 mg caffeine is administered to a subject and subsequent urinary or other bodily fluid analysis demonstrates the 3 demethylation derivative product of caffeine.

Methods or kits in accordance with the sixth or seventh aspects of the invention may be used as the basis of tests for cytochrome P450 2C9/10. Compounds known to undergo transformation by cytochrome P450 2C9/10 include: Amitriptyline, Diclofenac, Fluvastatin, Ibuprofen, Imipramine, Naproxen, Phenytoin, Piroxicam, Tolbutamide, Torasemide, Warfarin (Coumarin) and Zafirlukast. One or more of these compounds may be used as a prodrug in a method or kit in accordance with the present invention.

By way of example, a test for cytochrome P450 2C9/10 may involve administering a dose of 0.0001 mg to 50 gram Warfarin to a subject and testing the urine or other body fluid for the product of metabolism resulting from interaction of the substrate and enzyme system

It is particularly preferred that the methods or kits in accordance with the present invention be used in a test for the presence of Cytochrome P450 2D6. Such methods or kits may be used to assay for evidence for the metabolic activity of an enzyme to confirm whether that enzyme is present in an organism. In humans, for example, codeine is metabolised to morphine by the enzyme cytochrome P450 2D6.

It is known that up to 10% of the population do not express this enzyme and neither codeine nor morphine are naturally present in human body fluid. Therefore administering codeine (a prodrug in the context of the present invention) and subsequently testing for morphine (a metabolite produced on metabolism of morphine) will allow the inference that the enzyme cytochrome P450 2D6 is present in an individual so tested. If the metabolite is not found then it can be inferred that the enzyme is either inactive or absent, or perhaps that the test requires repeating with a higher dose of substrate.

An object of at least one aspect of this invention is to provide a means by which the presence of the enzyme CYP450 2D6 may be determined by untrained individuals in a reliable manner.

Accordingly the invention provides a kit comprising:
i) a supply of codeine (a prodrug), this supply being typically more than the minimum dose sufficient for enzymatic activity to produce detectable levels of morphine in the presence of active CYP450 2D6;
ii) means for testing for the presence of morphine; and optionally
iii) a test for the presence of codeine may also be provided (but is not essential for detecting the enzyme).

A preferred embodiment of the invention will now be described with reference to the accompanying drawing in which:

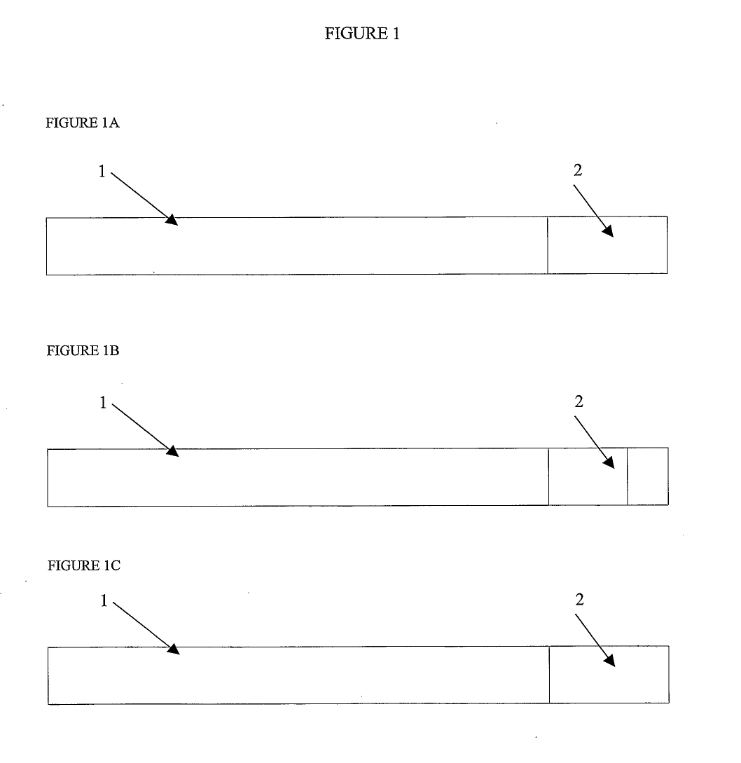

FIG. 1A shows a view of a test strip (a means for assaying for the presence of a metabolite) before use;

FIG. 1B shows a view of a test strip with a result confirming the presence of the metabolite morphine (and hence the presence in the subject of an effective amount of the enzyme cytochrome P450 2D6); and

FIG. 1C shows a view of a test strip with a negative result showing that cytochrome P450 2D6 enzyme activity is absent or insufficient to produce detectable levels of morphine.

### DETAILED DESCRIPTION OF A PREFERRED EMBODIMENT THE INVENTION

Before using the test administration of the enzyme substrate (prodrug) as directed is preferred otherwise a false negative result may be produced. The invention typically includes a supply of codeine to avoid this potential outcome.

As shown in FIG. 1A, the test strip comprises a length of material (1) to which is bonded a test strip (2) of paper or other device suitable for the detection of morphine or other metabolite. Such similar devices exist e.g. U.S. Pat. No. 3,901,657 but do not in themselves detect the presence of an enzyme. After consumption of the substrate and sufficient time for enzymatic activity the test strip is applied to a suitable body fluid, urine for example, and one of two results shown below may occur.

FIG. 1B, shows how the strip would appear in the presence of an active enzyme system. The length of material (1) remains unchanged but the test strip (2) shows detection of metabolite produces a colour or other change (in the drawing a new line is produced) indicating that the enzyme system is both present and sufficiently active for metabolite to be detected. In such a case a suitable analgesic regime may comprise the administration of codeine and/or tramadol.

FIG. 1C shows a negative result with the test strip for the metabolic product (2) test showing no colour or other change which allows the user to infer an absent or inactive enzyme system. In such a case a suitable analgesic regime may comprise the administration of dihydrocodeine.

A kit in accordance with the invention may also contain means (such as a similar test strip having suitable specificity) for detecting the substrate (prodrug). Though this is not essential for detecting the presence of the enzyme tested it would allow the user to assess whether sufficient substrate (prodrug) had been consumed in accordance with test procedure.

### EXPERIMENTAL RESULTS

The inventors assayed urine samples from ten patients for the presence of a metabolite indicative of metabolism of a prodrug. Prior to this assay each of the patients had been provided with a weak opioid analgesic as a prodrug.

The result obtained revealed that urine samples derived from three of the patients contained the metabolite, indicating that a suitable analgesic regime for alleviation of pain in these patients may comprise administration of at least one analgesic agent selected from the group consisting of: codeine and tramadol.

In contrast, urine samples from seven of the patients were negative for the metabolite. This indicated that a suitable analgesic regime for alleviation of pain in these patients should comprise administration of an analgesic agent selected from the group consisting of: dihydrocodeine and propoxyphene.

1. A method for selecting an analgesic regime for treatment of a patient, the method comprising assaying a body fluid sample from the patient for the presence of a metabolite produced by metabolism of a prodrug;
i) wherein presence of the metabolite in the body fluid sample indicates that a suitable analgesic regime for the patient should comprise administration of at least one analgesic agent selected from the group consisting of: codeine and tramadol; and
ii) wherein absence of the metabolite in the body fluid sample indicates that a suitable analgesic regime for the patient should comprise administration of an analgesic agent selected from the group consisting of: dihydrocodeine; buprenorphine and propoxyphene.

2. The method of claim 1, further comprising administering the prodrug to the patient prior to conducting the assay.

3. The method of claim 2, wherein the time elapsing between administration of the prodrug and assaying for the metabolite is less than a day

4. The method of claim 2, wherein the drug precursor is administered in an amount that is not sufficient to provide an analgesic effect.

5. The method claim 1, wherein the drug precursor comprises codeine

6. The method according to claim 5, wherein the codeine is administered in an amount between approximately 1 mg and 4 mg.

7. The use according to claim 5, wherein the metabolite is selected from the group consisting of morphine, morphine 3 gluceronide, and morphine 6 gluceronide.

8. The method of claim 1, wherein the drug precursor comprises tramadol.

9. The method according to claim 8, wherein the tramadol is administered in an amount between approximately 5 mg and 15 mg.

10. The use according to claim 8, wherein the metabolite is O-desmethyltramadol.

11. The method of claim 1, wherein the analgesic regime referred to in i) or in ii) further comprises administration of a non-opioid analgesic

12. The method of claim 1, wherein the analgesic regime referred to in ii) further comprises the administration of tramadol.

13. The method of claim 1, wherein the analgesic regime is for alleviation of pain associated with cancer.

14. The method of claim 1, wherein metabolism of the prodrug occurs via the action of a cytochrome P450 enzyme.

15. The method of claim 14, wherein the cytochrome P450 enzyme is cytochrome P450 2D6.

16. The method of claim 1, wherein the body fluid sample is selected from the group consisting of: a urine sample; a saliva sample; a blood sample; a plasma sample; a lymph sample; and a cerebrospinal fluid sample.

17. The method of claim 16, wherein the body fluid sample is a urine sample.

18. The method according to claim 1, wherein the assay for the metabolite is conducted by means of a reactive test device.

19. The method according to claim 1, wherein the assay for the metabolite is conducted by means of an immunoassay.

20. A method for selecting an analgesic regime for treatment of a patient, the method comprising assaying a body fluid sample from the patient for the presence of a metabolite produced by metabolism of a prodrug by cytochrome P450 2D6;
i) wherein presence of the metabolite in the body fluid sample indicates that a suitable analgesic regime for the patient should comprise administration of a prodrug metabolised by cytochrome P450 2D6 to yield an analgesic drug; and
ii) wherein absence of the metabolite in the body fluid sample indicates that a suitable analgesic regime for the patient should comprise administration of a drug having intrinsic analgesic activity.

21. A kit for selecting an analgesic regime for a patient, the kit comprising:
i) means for assaying for the presence of a metabolite produced by metabolism of a prodrug; and
ii) instructions for using the provided means to perform the method as described in claim 1.

22. A kit according to claim 21, further comprising a prodrug selected from the group consisting of: codeine and tramadol.

23. A kit according to claim 22, wherein the prodrug is provided in an amount of between approximately 1 mg and approximately 30 mg per test.

24. A kit according to claim 21, wherein the means for assaying for the presence of a metabolite comprise a reactive test device.

25. A kit according to claim 21, wherein the means for assaying for the presence of a metabolite comprise reagents for an immunoassay.

26. A kit according to claim 25 comprising an antibody specific for the metabolite.

27. A kit according to claim 21, comprising means for assaying for the presence of a metabolite selected from the group consisting of: morphine; morphine 3 gluceronide; morphine 6 gluceronide; and O-desmethyltramadol.

28. The use of a means for detecting a metabolite produced by the action of a cytochrome P450 on a prodrug in the manufacture of a kit for the selection of an analgesic regime for a patient.

29. A method for selecting an analgesic regime for treatment of a patient, the method comprising assaying a sample representative of gene expression in the patient for an expression product representative of expression of cytochrome P450 2D6;
i) wherein presence in the sample of the expression product representative of expression of cytochrome P450 2D6 indicates that a suitable analgesic regime for the patient should comprise administration of codeine and/or tramadol; and
ii) wherein absence in the sample of the expression product representative of expression of cytochrome P450 2D6 indicates that a suitable analgesic regime for the patient should comprise administration of an analgesic agent selected from the group consisting of: dihydrocodeine; buprenorphine and propoxyphene.

30. The method of claim 29, wherein the sample contains cells from which information regarding gene expression may be derived.

31. The method of claim 29 wherein the cells are obtained by means of a swab.

32. The method of claim 29, wherein the expression product representative of expression of cytochrome P450 2D6 comprises a polypeptide.

33. The method of claim 29, wherein the expression product representative of expression of cytochrome P450 2D6 comprises a nucleic acid molecule encoding cytochrome P450 2D6.

34. A kit for selecting an analgesic regime for treatment of a patient, the kit comprising:
i) means for assaying a sample representative of gene expression in the patient for an expression product representative of expression of cytochrome P450 2D6;
ii) and instructions for using the means provided to perform the method as described in claim 29.

35. A kit according to claim 34, wherein the means comprise an antibody, or antibody fragment, capable of binding specifically to a polypeptide expression product.

36. A kit according to claim 34, wherein the means comprise primers for the amplification of a nucleic acid expression product.
